(19) United States
(12) Patent Application Publication
(10) Pub. No.: US 2002/0032412 A1
(43) Pub. Date: Mar. 14, 2002

(54) Syringe with retractable needle and a single use syringe

(76) Inventor: John Riemelmoser, Salisbury Heights (AU)

Correspondence Address: ATTN: Michael H. Jester, THE LAW OFFICES OF MICHAEL H. JESTER, SYMPHONY TOWERS, 750 B STREET, SUITE 256, SAN DIEGO, CA, 92101, US

(21) Appl. No.: 09/935,391

(22) Filed: Aug. 22, 2001

(30) Foreign Application Priority Data
Aug. 24, 2000 (AU) PQ 9619
Oct. 10, 2000 (AU) PR 0676

(51) Int. Cl. A61M 5/32

(52) U.S. Cl. 604/197

(57) ABSTRACT

This invention relates to a syringe (10) and a sleeve (11) assembly wherein the needle (13) of the syringe (10) is retractable with respect to the sleeve (11). The sleeve (11) has an open end (18) and a closed end (19) with an aperture (20) through the closed end (19). A syringe (10) with a needle (13) located within the sleeve (11) is movable between a first position, in which the needle (13) is within the sleeve (11), and a second position where the needle (13) is located through the aperture (20) so that it protrudes from the closed end (19) of the sleeve (11). A biasing element (22) acts between the syringe (10) and the sleeve (11) and applies a retraction force to move the syringe (10) from the second position to the first position so as to withdraw the needle (13) from the closed end (19) of the sleeve (11). The invention allows automatic and failsafe retraction of the needle (13) once the syringe (10) and sleeve (11) assembly is discarded.

This invention relates to a syringe with a needle protection system and in particular to an arrangement that ensures that the needle retracts into a container to ensure that it is totally encapsulated to thereby prevent needle stick injuries.

The invention also relates to a single use syringe design.

### BACKGROUND OF THE INVENTION

Needle stick injuries resulting from contact with discarded needles is an increasing problem. The handling of syringes by medical personnel also exposes them to accidental needle stick injuries.

It is the object of this invention to provide a means whereby the needle of the syringe is retracted to a position where it is shielded to avoid the potential for needle stick juries.

### BRIEF DESCRIPTION OF THE INVENTION

One aspect of the invention is a syringe assembly with a retracting needle comprising:

a sleeve having an open end and a closed end,

an aperture through said closed end,

a syringe with needle located within said sleeve that is movable between a first position where said needle is within said sleeve and a second position where said needle is located through said aperture so that it protrudes from the closed end of said sleeve, and

a biasing element acting between said syringe and said sleeve that applies a retraction force to move said syringe from said second position to said first position.

The term “closed end” is used to indicate, not that the end is fully closed off, but that the end forms an effective barrier against unwanted exposure of the needle. The aperture through the closed end is only large enough to allow the needle to be located therethrough with a small amount of care first being required to align the needle with the aperture to allow the syringe to be pushed downwardly against the biasing element to therefore expose the needle from the end of the sleeve.

Preferably, the biasing element, in addition to applying a force along the longitudinal axis to move the syringe to its first position, also applies a small sideways load to ensure that the syringe is pushed to one side. This will cause the needle to be positively pushed out of alignment with the aperture to prevent unwanted exposure of the needle.

Preferably, there is a retaining means between the syringe and the sleeve which prevents the syringe from being removed from the open end of the sleeve. This may comprise a longitudinal slot within the sleeve and a projection on the syringe that engages within the slot. The biasing means acts to push the syringe so that the projection abuts against the upper end of the longitudinal slot. This acts to hold the syringe within the sleeve.

Once the needle is aligned with the aperture, the syringe can be pushed downwardly within the sleeve against the biasing element so that the needle projects from the end of the sleeve. Downward movement of the syringe is limited either by the needle hub bearing against the inside surface of the sleeve or the projection bearing against the lower end of the longitudinal slot.

Preferably, the means used to secure the syringe within the sleeve should be such that any attempts to remove the syringe from the sleeve will damage it to the extent where it would not be serviceable. The projection may be designed to allow the syringe to be located into the sleeve for assembly purposes, but may be such that any attempt to disassemble it will in turn fracture the syringe body. This could be achieved by providing an area of reduced thickness around the projection which is weakened sufficiently to fracture upon forced removal.

Once the syringe has been used, the biasing element acts to push the syringe upwardly so that the needle retracts from the end of the sleeve. This will occur once the syringe is released with respect to the sleeve. Preferably this occurs automatically as soon as the user of the syringe assembly releases the end of the syringe. To this end, the sleeve may further comprise finger flanges that are gripped while the plunger of the syringe is depressed. Release of the plunger will result in the biasing element pushing the syringe upwardly thereby retracting the needle with respect to the end of the sleeve. A catch between the syringe and the sleeve may be used to hold the syringe body so that the needle remains exposed from the end of the sleeve prior to use of the syringe. The catch is preferably automatically released after use of the syringe. The side load applied to the syringe will result in the needle being placed out of alignment of the aperture once it is fully within the sleeve.

An object of a second aspect of this invention is to provide a syringe which is only capable of being used once.

Accordingly, a second aspect of the invention is a single use syringe comprising:

a syringe barrel,

a plunger for use in said syringe barrel, and

a plunger bulb releasably attached to said plunger so that said plunger will push said plunger bulb into said barrel but will detach from said plunger bulb as said plunger is withdrawn from said barrel.

In known syringes, it is common to have a connection between the plunger and plunger bulb which will enable the plunger to pull the plunger bulb towards the open end of the syringe barrel as well as pushing it back towards the needle end. However, in this aspect of the invention, the plunger does not connect to the plunger bulb in this manner but does so in a manner which prevents the plunger bulb from being drawn back towards the open end of the syringe barrel.

In order to use the syringe in this manner, the syringe is supplied with the plunger and plunger bulb located external of the syringe barrel. The syringe is loaded with the liquid to be injected and then the plunger and plunger bulb are inserted and pushed into the barrel to first exclude air, and then to secondly inject the liquid. The plunger bulb will be left at the end of the syringe once the plunger is pulled back and releases from the plunger bulb.

Accordingly, the end of the plunger which engages with the plunger bulb may be a plain shaft which locates within an aperture that does not in any way grip that part of the plunger. This would enable the plunger bulb to be pushed in one direction but not pulled back.

Preferably, the syringe barrel may be provided with a funnel or similar means to enable liquids to be easily poured into the syringe barrel. This would be particularly necessary in the case of low volume syringes which are quite long and therefore reasonably small in diameter.

The second aspect of the invention could be used with the first aspect of the invention so that a single use syringe could be provided with a needle retracting feature.

A third aspect of the invention is a single use syringe comprising:

a syringe barrel,

a plunger for use in said syringe barrel,

a plunger bulb releasably attached to said plunger, and

a locking mechanism within said syringe barrel located towards the discharge end of said syringe barrel that engages and holds said plunger bulb, said plunger being able to push and pull said plunger bulb within said barrel while said locking mechanism is not engaged and said plunger detaching itself from said plunger bulb if said plunger is withdrawn from said barrel once said plunger bulb is engaged with and held by said locking mechanism.

This aspect of the invention differs from the second aspect of the invention in that the plunger and plunger bulb can be moved back and forth along most of the syringe barrel. Provided that the plunger bulb does not engage the locking mechanism, then the plunger will remain engaged with the plunger bulb and enable it to be moved back and forth within the syringe barrel.

This will enable fluid to be drawn into the syringe, and air to be expelled from the syringe. As well, drawing back on the plunger after the syringe needle is inserted into a patient will ensure that the needle is correctly located within a vein.

Preferably, the plunger bulb engages the locking mechanism after the plunger is fully depressed. This then means that the plunger bulb is held captive by the locking means after the syringe has been used. Withdrawal of the plunger once the plunger bulb is engaged with the locking mechanism will result in release of the plunger thereby disenabling the syringe.

Preferably, the locking mechanism comprises at least one internal projection within the syringe barrel which the plunger bulb can pass but applies a holding force which exceeds the retaining force between the plunger bulb and plunger when an attempt is made to pull the plunger bulb past the projection.

The connection between the plunger bulb and plunger is designed to release prior to overcoming the retaining force applied to the plunger bulb by the locking mechanism.

Preferably, a plurality of internal projections are used, and these internal projections may be formed by applying deformation force to the external surface of the syringe barrel. This force is sufficient to form permanent projections on the internal surface of the syringe barrel which, in operation of the syringe can be passed by the plunger bulb, but which provides sufficient holding force to result in the plunger being detached from the plunger bulb without the plunger bulb releasing from the locking mechanism.

In order to fully understand the invention, a preferred embodiment will now be described. However, it should be realised that the scope of the invention is not to be confined or limited to this preferred embodiment. The preferred embodiment is illustrated in the accompanying representations in which:

### DESCRIPTION OF THE DRAWINGS

FIG. 1 shows a cross-sectional view of a sleeve,

FIG. 2 shows a syringe and needle with a fitted spring to be located within the sleeve shown in FIG. 1,

FIG. 3 shows a cross-sectional view of a sleeve with a syringe located in the sleeve so that the needle projects through an aperture in the sleeve,

FIG. 4 shows the syringe in a retracted position in the sleeve so that the needle of the syringe is contained within the sleeve,

FIG. 5 shows the plunger of the syringe being withdrawn and releasing from the plunger bulb,

FIGS. 6 to 8 show a cross sectional view of a syringe in accordance with a third embodiment where the syringe body includes a locking mechanism, and

FIG. 9 shows a prior art cross sectional view of a syringe according to a third embodiment showing the release of a plunger from the plunger bulb.

### DETAILED DESCRIPTION OF EMBODIMENTS OF THE INVENTION

This embodiment comprises an assembly of a syringe 10 with a sleeve 11. The syringe 10 has a syringe barrel 12, a needle 13, a plunger 14 and a plunger bulb 15.

The sleeve 11 has an opened end 18 and a closed end 19. The closed end 19 has an aperture 20 through the closed end 19. The aperture 20 is small, but is of sufficient diameter to allow the needle 13 to be located within the aperture 20. It can then be moved through the aperture 20 to expose the needle 13 from the end of the sleeve 11.

In this embodiment, the biasing element comprises a spring 22 fitted around the barrel 12 of the syringe 10. Other forms of biasing means may be used and could include an element that is integrally moulded to the barrel 12 of the syringe 10.

A projection 24 is located on the external surface of the barrel 12. The projection 24 locates within a slot 25 formed in the wall of the sleeve 11. The projection 24 when located within the slot 25, prevents rotation of the syringe 10 with respect to the sleeve 11 and also prevents the syringe 10 from being pushed out of the opened end 18 of the sleeve 11 by the spring 22.

The spring 22 is held between the projection 24 on the syringe 10 and a projection 26 on the internal surface of the sleeve 11. The spring 22 is compressed when the needle 13 is aligned with and inserted through the aperture 20. The needle 13 remains exposed from the end of the sleeve 11 while the spring 22 is held in its compressed state. Holding the barrel 12 with respect to the sleeve 11 or depressing the plunger 14 will hold the spring 22 in its compressed state so that the needle 13 remains exposed from the end of the sleeve 11.

Releasing the syringe 10 with respect to the sleeve 11 will result in immediate movement of the syringe 10 with respect to the sleeve 11. The compression in the spin 22 will push the syringe 10 upwardly so that the needle 13 retracts into the sleeve 11. Preferably there is no catch or lock between the syringe 10 and the sleeve 11 that removes the need to hold the syringe 10 with respect to the sleeve 11 to keep the needle 13 in an exposed position. Preferably, the needle 13 will automatically retract as soon as the syringe 10 is released. This avoids the need for any manual action to release a catch between the syringe and the sleeve 11.

However, the provision of such a catch or lock between the syringe 10 and the sleeve 11 would still be within the scope of this invention.

When the needle 13 is in its retracted position as shown in FIG. 4, the spring 22 preferably applies a side load to the syringe 10 which causes the needle 13 to be moved out of alignment with respect to the aperture 20. Further, the aperture 20 is sufficiently small in size to prevent accidental exposure of the needle 13. The needle 13 has to be aligned with a reasonable degree of precision with the aperture 20 in order to allow the needle 13 to be exposed from the end of the sleeve 11.

A second aspect of the invention is the design of the plunger 14 and plunger bulb 15 which allow the syringe 10 to be used only once. As seen in FIG. 5, the end of the plunger 14 is plain and is not provided with a barb or an enlarged end which is normally used to secure the plunger 14 to the plunger bulb 15. Also, the plunger bulb 15 has a plain aperture within which the end of the plunger 14 locates. This means that the plunger 14 can only push the plunger bulb 15 into the syringe 10. The plunger 14 is immediately released from the plunger bulb 15 when it is withdrawn and the plunger bulb 15 is left in the barrel 12 of the syringe 10.

The syringe barrel 12 is provided with a funnel 28 which enables the liquid to be poured into the barrel 12 prior to insertion of the plunger 14 and plunger bulb 15. Accordingly, instead of drawing liquid into the syringe 10 by pulling the plunger 14 out of the syringe, the barrel 12 is first filled before inserting the plunger 14.

The syringe 10 can then be inverted so that the plunger bulb 15 can be pushed into the barrel 12 to vent the air space. The injection can then take place whereupon the plunger bulb 15 is pushed to the end of the barrel 12. Removal of the plunger 14 will leave the plunger bulb 15 in the position shown in FIG. 5.

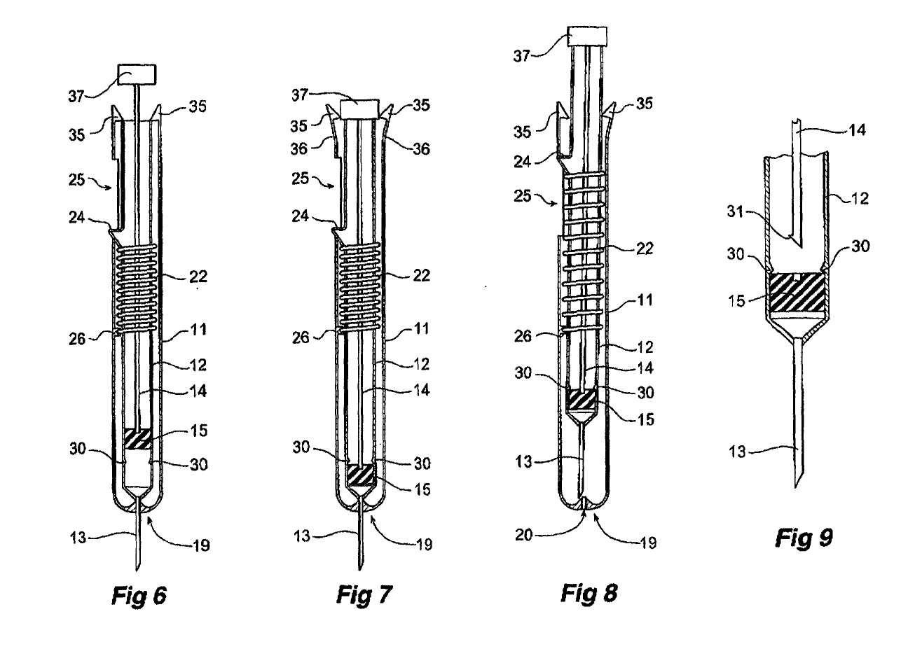

A third aspect of the invention is an alternate means of ensuring single use of the syringe 10. Accordingly, a third embodiment is illustrated in FIGS. 6 to 9 which provide a plunger 14 and plunger bulb 15 which are able to move to a certain extent within the barrel 12 while at the same time ensuring that the syringe 10 becomes unusable once the plunger bulb 15 is fully depressed.

In this embodiment, the barrel 12 is provided with a plurality of projections 30 that are formed on the internal surface of the barrel 12 towards the discharge end of the syringe 10. The connection between the plunger 14 and plunger bulb 15 is such that it can withstand a sufficient withdrawal force being applied to the plunger 14 so that the plunger bulb 15 can be pulled out of the barrel 12.

However, this connection between the plunger 14 and plunger bulb 15 is such that when engaged by the projections 30 of the locking mechanism, the force required to withdraw the plunger bulb 15 exceeds the connection force between the plunger 14 and the plunger bulb 15. This results in the plunger 14 pulling free of the plunger bulb 15 as illustrated in FIG. 9. The connection between the plunger 14 and the plunger bulb 15 in this embodiment comprises a conventional barb 31 which locates within an aperture in the plunger bulb 15. The size of the barb 31 and the fit of the barb 31 within the aperture and the plunger bulb 15 is such that the components will separate once a required force is applied.

In this embodiment, the projections 30 are formed within the barrel 12 by applying an external deformation force to the barrel 12 during the manufacturing process. The barrel 12 in this embodiment is a moulded plastic component, and provided that a point load is applied to the barrel while it is still hot and reasonably pliable, then a permanent deformation will result.

This process could occur directly after moulding of the barrel 12, or may occur at a later stage with localised heating being applied to the barrel 12.

The number of projections 30 and the extent to which they project with the barrel 12 can be varied to achieve the required retaining force. Obviously, this force will as least need to exceed the maximum retaining force between the plunger 14 and the plunger bulb 15.

The projections 30 of the locking mechanism are located towards the discharge end of the syringe 10. This enables the syringe 10 to be operated in a conventional manner in that, unlike the second embodiment, the plunger 14 can be used to draw fluid into the syringe 10. Obviously withdrawal of the plunger bulb 15 will commence above the projections 30. The included air within the syringe 10 can be excluded in the normal manner by inverting the syringe 10 and expelling the air by depressing the plunger 14.

The plunger 14 can then be slightly withdrawn when it is inserted within a person to ensure that the needle 13 is correctly located within a vein. Withdrawal of the plunger 14 will result in a tell-tail stream of blood becoming visible within the syringe 10 if the needle 13 is correctly located.

The plunger 14 can then be fully depressed to inject the fluid from the syringe 10. This will result in the plunger bulb 15 engaging the projections 30 of the locking mechanism. Any further retraction force applied to the plunger 14 will result in it's disconnection from the plunger bulb 15. The syringe 10 according to this third embodiment will be a single use syringe provided that the plunger bulb 15 engages the projections 30 of the locking mechanism.

The plunger 14 and plunger bulb 15 according to this third embodiment will obviously, as is the case with the second embodiment, be equally suited to use with the sleeve 11 to retract the needle 13 to provide protection against needle stick injuries.

In a further embodiment of the invention, a catch is provided between the sleeve 11 and the barrel 12 so that the spring 22 remains in a compressed state without the user having to hold the barrel 12 with respect to the sleeve 11. This will result in the needle 13 being held in its exposed position from the end of the barrel 12.

As shown in FIGS. 6, 7 and 8 a catch comprises a pair of projections 35 that locate over the end of the barrel 12. The projections 35 are on the end of arms 36 that are pivotally attached to the sleeve 11. In this embodiment, the arms 36 and projections 35 comprise components that are integrally moulded together with the sleeve 11. The arm 36 hinge due to the flexibility of the material used to mould the sleeve 11. While the projections 35 remain engaged over the end of the barrel 12, the needle 13 remains in the position shown in FIG. 6 where it extends from the end of the sleeve 11. The syringe can then be filled and prepared for injecting.

When the plunger 14 is fully depressed, the end of the plunger 37 abuts against the projections 35 and spreads the arms 36 as shown in FIG. 7. The plunger 14 must be fully depressed to this position in order to empty the syringe 10 to the maximum possible extent. The end of the plunger 37 spreads the arms 36 so that the projections 35 disengage from the end of the barrel 12. In this position, the barrel 12 is no longer retained and the spring 22 pushes the barrel 12 outwardly so that the needle 13 retracts within the sleeve 11.

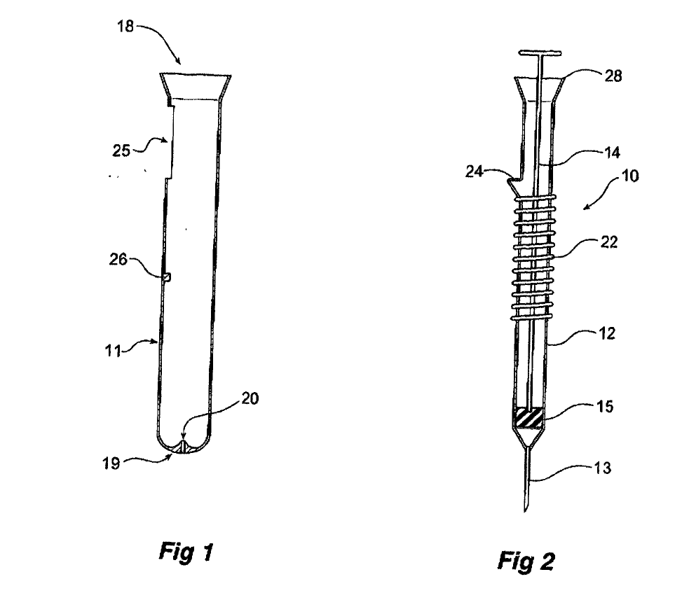

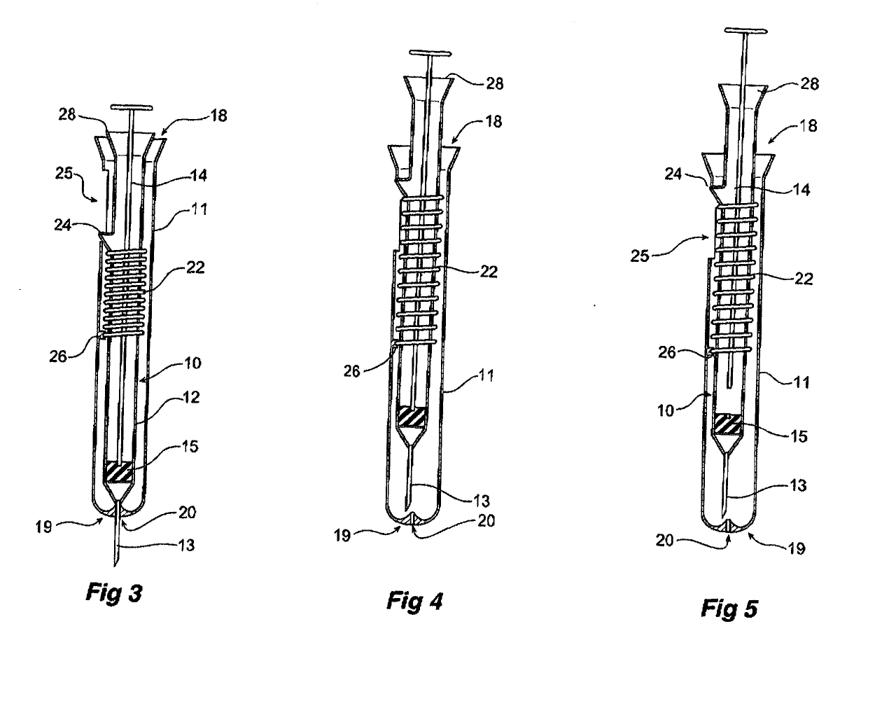

This catch between the barrel 12 and sleeve 11 is illustrated in FIGS. 6, 7 and 8 in combination with the third embodiment. However, the catch means shown in these Figs. would be equally suited to the embodiments illustrated in FIGS. 1 to 5.

As will be seen from the above description, the various aspects of this invention provide a syringe assembly which is particularly useful for small volume syringes 10. They also ensure a fail safe means of preventing exposure to syringe needles 13. Unlike other retractable needle systems, there is no positive action required by the user to ensure retraction of the needle 13.

In addition, the invention provides methods of preventing multiple use of a used syringe.

The claims defining the invention are as follows:

1. A syringe and sleeve assembly wherein the needle of said syringe is retractable with respect to said sleeve comprising:
a sleeve having an open end and a closed end,
an aperture through said closed end,
a syringe with needle located within said sleeve that is movable between a first position, in which said needle is within said sleeve, and a second position where said needle is located through said aperture so that it protrudes from the closed end of said sleeve, and
a biasing element acting between said syringe and said sleeve that applies a retraction force to move said syringe from said second position to said first position.

2. A syringe and sleeve assembly according to claim 1 wherein said biasing element also applies a side load to said syringe to push said needle away from alignment with said aperture.

3. A syringe and sleeve assembly according to either claim 1 or 2 further comprising a retaining means between said syringe and said sleeve that holds said syringe within said sleeve.

4. A syringe and sleeve assembly according to claim 3 wherein said retaining means comprises a projection on said syringe and a slot within said sleeve within which said projection locates.

5. A syringe and sleeve assembly according to claim 4 wherein said projection is connected to said syringe by a region of weakness that will fracture upon attempting to remove said syringe from said sleeve, where said fracturing renders said syringe unusable.

6. A syringe and sleeve assembly according to claim 1 wherein said biasing element comprises a spring acting between said syringe and sleeve.

7. A syringe and sleeve assembly according to claim 6 wherein said spring comprises a helical spring located around said syringe with one end of said helical spring secured to said syringe and the other end of said helical spring secured to said sleeve.

8. A syringe and sleeve assembly according to claim 1 wherein the plunger and plunger bulb of said syringe are releasably attached so that said plunger will push said plunger bulb into said syringe but will detach from it as said plunger is withdrawn from said syringe.

9. A syringe and sleeve assembly according to claim 8 wherein the end of said plunger is plain and said plunger bulb has an aperture into which said plunger releasably locates.

10. A syringe and sleeve assembly according to claim 9 wherein the end of said syringe is enlarged in diameter so as to form a funnel.

11. A syringe and sleeve assembly according to claim 1 wherein the plunger and plunger bulb of said syringe are releasably attached to enable said plunger to push and pull said plunger bulb within said syringe but to release when said plunger bulb is retained.

12. A syringe and sleeve assembly according to claim 11 wherein said syringe further comprises a locking mechanism within said syringe located towards the discharge end of said sage that engages and holds said plunger bulb so that said plunger is released from said plunger bulb when withdrawal force is applied.

13. A syringe and sleeve assembly according to claim 12 wherein said locking mechanism comprises at least one internal projection within said syringe that said plunger bulb passes or engages to thereby be held with respect to said syringe.

14. A syringe and sleeve assembly according to claim 1 further comprising a catch on said sleeve that holds said syringe in said second position, said catch being automatically released upon the plunger of said syringe being fully pushed into said syringe to thereby release said syringe so that it is free to move into said first position.

15. A syringe and sleeve assembly according to claim 14 wherein said catch comprises a pair of arms and projections on said arms that locate over the end of said syringe.

16. A syringe and sleeve assembly according to claim 15 wherein the end of said plunger abuts against said projections and causes said arms to be deflected outwardly to thereby release said catch.
